(19) United States
(12) Patent Application Publication
(10) Pub. No.: US 2017/0056095 A1
(43) Pub. Date: Mar. 2, 2017

(54) Work Device For A Microsurgical Instrument, Microsurgical Instrument, And Power And Torque Transmission Hose

(71) Applicant: Karl Storz GmbH & Co. KG, Tuttlingen (DE)

(72) Inventors: Jochen Stefan, Wald (DE); Dominik Volkmer, Fridingen (DE)

(21) Appl. No.: 15/238,694

(22) Filed: Aug. 16, 2016

(30) Foreign Application Priority Data
Aug. 26, 2015 (DE) 102015010970.7

(51) Int. Cl. A61B 18/14; A61B 17/29

(52) U.S. Cl. CPC A61B 18/1445; A61B 17/2909; A61B 2017/00477

(57) ABSTRACT

A work device for a microsurgical instrument that includes a proximal coupling portion which can be coupled with a handle. A hollow shaft extends from the coupling portion to a distal tool end on which is positioned a jaw-shaped work apparatus, the work apparatus having two arms which can pivot with respect to one another, and the work apparatus being mounted so that it can rotate about the longitudinal axis of the shaft. In the interior of the shaft a power and torque transmission hose runs from the coupling portion to the distal tool end and is operatively connected with the work apparatus. The power and torque transmission hose is made rigid in at least one portion, situated between the coupling portion and the distal tool end, by at least one pulling element running in the longitudinal direction of the power and torque transmission hose.

The following invention relates to a work device for a microsurgical instrument, to the microsurgical instrument itself, and to a power and torque transmission hose for the work device.

### TECHNICAL FIELD

Minimally invasive surgical methods, to an increasing degree, are replacing classical open surgical techniques, resulting in advantages that, in addition to a better cosmetic outcome (a shorter skin suture), include, in particular, reduced damage to surrounding tissue and thus briefer hospital stays. Along with purely mechanical instruments with jaw-shaped tools, such as scissors, forceps, needle holders, etc., electrosurgical instruments are also well known for microsurgery. Thus, between the tissue that is to be cut and/or held, high-voltage current (HF tension) is applied, resulting in a stop to bleeding (coagulation) by warming the surrounding tissue.

### BACKGROUND

An electrosurgical instrument is disclosed, for example, in DE 602 26 015 T2.

The tool ends of such electrosurgical instruments are intended to possess, besides the coagulation function, all the functions of classic microsurgical instruments known in this field, such as flexibility for pivoting and/or bending as well as good force feedback.

Construction space available inside the shaft in commonly known shaft diameters has a width of just a few millimeters. The solution has been to convert to using a combined power and torque transmission element, by which it is possible to control both the opening and closing movement of the arms as well as a rotating motion of the work device. It is customary here, in addition to rigid tubes in bendable tube segments, to use a power and torque transmission tube having torsion rigidity. A microsurgical instrument employing a hose as power transmission element is disclosed in EP 2837340 A1.

In such microsurgical instruments, however, the maximal actuation powers achievable on the arms are restricted by elastic deformations of the power and torque transmission hose, which is unsatisfactory for the operator, in particular in grasping thicker tissue parts. In addition, with electro-surgical instruments constructed on this principle, regulation of the flow density applied for coagulation is not possible with precision, because this regulation of the flow density applied to coagulation occurs essentially by way of the pressure exerted on the gripped tissue (at stronger pressure, a lesser transition resistance arises and vice versa). To be able to regulate the amount of flow density as precisely as possible, therefore, requires good force feedback, which is impeded by elastic deformation, that is, insufficient rigidity of the hose.

### SUMMARY

On the basis of this prior art, it is the object of the present invention to provide an improved work device for a microsurgical instrument, in particular to make possible greater actuation forces on the arms and to allow improved force feedback and, when configured as an electrosurgical instrument, improved capability of regulating current density.

This object is fulfilled by means of a work device for a microsurgical instrument according to the present invention.

In addition, there is the object of providing a microsurgical instrument by which increased actuation forces can be achieved and which allows good force feedback from the surgical area and is simple to operate.

This object is fulfilled by means of a microsurgical instrument according to the present invention.

Finally, there is also the object to provide a power and torque transmission hose by which existing work devices can be re-engineered and thereby improved in the manner described above.

This object is achieved by means of a power and torque transmission hose according to the present invention.

Preferred refinements are described in each case in the subsidiary claims.

The inventive work device for a microsurgical instrument comprises, in a first embodiment, a proximal coupling portion, which can be coupled with a handle. From the coupling portion, a hollow shaft extends to a distal tool end, on which a jaw-shaped work device, rotatably mounted around the longitudinal axis of the shaft, is positioned, with at least two arms that can pivot toward one another. In the interior of the shaft, a power and torque transmission apparatus extends from the coupling portion to the distal tool end and is operatively linked with the work apparatus by opening and closing forces in order to transmit a rotary motion. Said power and torque transmission apparatus is configured, at least along one longitudinal portion, as a power and torque transmission hose, which is stiffened in at least one longitudinal portion by at least one pulling element running in the longitudinal direction of the power and torque transmission hose.

The phrase “at least one portion” in the foregoing can be taken to mean one connected portion or else two or more separate portions. The portion of the power and torque transmission apparatus configured as the power and torque transmission hose can, for example, be situated in a bendable portion of the shaft, because the hose is flexible and follows the bend. However, it may occur that the power and torque transmission apparatus over its entire length consists of the power and torque transmission hose. The part of the power and torque transmission apparatus that is not configured as a hose can be, for example, a rigid tube.

The term “work device,” meaning a workable implementation, is understood here as part of a microsurgical instrument that comprises the shaft and the jaw-shaped work apparatus on the distal end (scissors, gripper jaw, needle holder, or the like). The work device as understood by the present invention also includes one or more power and/or torque transmission means, which run in the shaft and with which an actuation force can be exerted upon the distal work apparatus by an actuation element of the handle. The work device is coupled with the handle in order to provide a workable microsurgical instrument.

The work apparatus can, for example, be turned about the longitudinal axis of the shaft at an angle between 0 and 90°, 0 and 180° or still greater or even unlimited.

The power and torque transmission hose should be configured, as far as possible, to be resistant to torsion, so that rotation commands can be transmitted as directly as possible and without distortions to the work apparatus. Practitioners in the art know how to construct a power and torque transmission hose which has these characteristics (compare the principle of a bendable axle or of a shower hose).

The shaft of the inventive work device has, for instance, a diameter of a few millimeters, typically about 2.5 to 8 mm; greater or smaller formats are also possible.

Since the invention foresees reinforced pulling of the power and torque transmission device, its pulling rigidity is substantially increased, leading to lesser deformations and increasing the forces that can be exerted on the arms of the work apparatus while improving the force feedback. With an inventive work device of a particular diameter, it thus becomes possible to sever or to grip considerably thicker tissues, and/or to exert higher suturing forces than with a known work device of equal diameter class. Changes in construction according to the invention advantageously affect only the power and torque transmission hose in itself, while leaving the other components of the work device unchanged; thus it is possible to achieve a comparatively large gain in useful value by means of a structural adjustment that is surprisingly minor and therefore cost-effective.

The arms of the work apparatus can each comprise an effective segment in the vicinity of their distal ends, preferably a blade or gripping surface; the work device therefore, depending on the configuration of the effective segments, can take the form, for instance, of a needle holder, forceps, scissors, biopsy forceps, spreading forceps or dissection forceps.

It is possible then that one of the arms remains stationary and the other is movable, or that both arms are movable and are pivotably mounted in a separate base of the work apparatus. It is possible here to ensure that the power and torque transmission hose is connected with the base in order to trigger the rotary movement.

In an additional embodiment, the pulling element can be positioned either inside the power and torque transmission hose, such as on the inner diameter, or outside the power and torque transmission hose, such as on the outer diameter. In addition, the pulling element can be connected, preferably welded, with the power and torque transmission hose, at least in a proximal and a distal end region of the power and torque transmission hose. The power and torque transmission hose can take the form, for example, of a rope, such as a metallic rope, a wire, a fiber or fiber bundle.

If the pulling element is mounted in the power and torque transmission hose, the outer dimensions as well as the connecting areas are advantageously retained and the use of the stiffened power and torque transmission hose requires no rebuilding of the work device. Since the pulling element extends over the full length of the power and torque transmission hose and is connected only in the respective end portions, the power and torque transmission hose is released in the essential portion of its length from the transmission of pulling forces, resulting in substantially lesser distensions, so that the force feedback characteristic are improved and the maximum gripping force is increased.

In yet another embodiment, the work apparatus can be connected with the shaft by means of a joint by which the work apparatus can be turned about a rotary axis running perpendicular to the longitudinal axis of the shaft. Alternatively or in addition, the shaft can comprise in a bendable shaft segment at least one joint, in particular two or more joints, by which the shaft segment can be turned about a rotary axis running perpendicular to the longitudinal axis of the shaft. In the bendable shaft segment, the power and torque transmission apparatus can advantageously be configured as a power and torque transmission hose to enable it to follow the bending motion. The power and torque transmission hose can, in particular, be fed through the joints. In addition, the shaft can be configured as bendable in one or more longitudinal portion(s).

In addition, the work device can take the form of an electro-surgical work device, with one or more electrical connection line(s) which extend from the coupling segment to the distal tool end and which are electrically connected with one or both arms. The term “electrically connected” includes a direct connection with the arms as well as an indirect connection by interposed transmission elements. Whether one or two electrical connection lines are foreseen depends on whether the instrument is configured as unipolar or bipolar. In the case of a bipolar instrument, each connecting line is connected with an arm, while in the case of a unipolar instrument only one of the arms is in contact with the electrical connecting line while the other is connected with the neutral electrode, which in turn during its application is connected over the largest possible surface with the patient's body. The skilled practitioner is aware of the connecting lines that are to be installed; he will select them depending on the strength of the current, voltage and the arising strengths of the HF tension.

It is possible to provide for the electrical connecting line or the two connecting lines to run inside the power and torque transmission hose. In particular, it can be arranged that the connecting line(s) are positioned on a peripheral position of the power and torque transmission hose situated opposite the pulling element or else run centrally. Above all, care must be exercised to ensure a sufficient distance between the pulling element and the electrical connection, so that the electrical connecting line and/or the covering/insulation is not damaged because of relative movements.

The inventive microsurgical instrument comprises, in a first embodiment, a proximal handle with at least one actuation element and a distal inventive work device, which is operatively coupled via its coupling portion by a corresponding coupling device of the handle. The work device here can be connected with the handle in a choice of either permanent or replaceable manner.

The handle can be, for example, a pistol handgrip or pincer grip on which two mechanical actuation elements are positioned: a first actuation element for exerting torque on the power and torque transmission apparatus of the work device, and a second actuation element for exerting a force in the longitudinal direction of the power and torque transmission apparatus of the work device. The handle can also, for example, have a reset apparatus to replace the actuation apparatus(es) in an at-rest position. One or both of the mechanical actuation elements can be a manual actuation element, such as a rotary wheel, a lever, a pushbutton. Alternatively or in addition, one or both of the mechanical actuation elements can be an actuation element by which a power drive apparatus can be activated that is configured to act upon the power and torque transmission hose of the work device. The power drive apparatus can, for example, be a rotary and/or linear drive, such as an electrical or pneumatic rotary and/or linear power drive.

If the rotary power drive of the handle is used to generate torque for the rotary motion of the work apparatus around the longitudinal axis (=rollers) of the shaft, the work apparatus can advantageously be rotated without limit about the longitudinal axis of the shaft, without having to wind it back, something that is not necessarily possible in manual actuation.

In configuring the microsurgical instrument as an electro-surgical instrument, the handle can be connected with a power source, such as an HF tension source. The handle then usefully has an electrical coupling apparatus, which is connected or connectable with an electrical junction of the work device. It is also possible, in addition, that an electrical actuation element is present on the handle in order to activate and/or deactivate a flow of current from the power source to the work device.

The inventive power and torque transmission hose for a work device of a microsurgical instrument, according to a first embodiment, is made rigid in at least one portion situated between the two ends with at least one pulling element running in the longitudinal direction of the power and torque transmission hose. The rigidity in the pulling direction is hereby increased, so that a work device and/or microsurgical instrument, in which the power and torque transmission hose is inserted, makes greater working forces possible and thus becomes more comfortable to operate because of improved force transmission properties. The pulling element here can extend over the entire length of the power and torque transmission hose or only in one partial segment.

In an additional embodiment of the power and torque transmission hose, the pulling element can be positioned inside the power and torque transmission hose, such as on the inner diameter, or outside the power and torque transmission hose, such as on the outside diameter. With the arrangement in the power and torque transmission hose, the outer dimensions and the adjoining areas advantageously remain available for force insertion and expulsion, so that the inventive power and torque transmission hose can be employed one to one as a replacement in back-up instruments.

Alternatively or in addition, the pulling element can be connected in a proximal and distal end portion of the power and torque transmission hose with the power and torque transmission hose, for instance directly welded. It is not impossible, however, for the pulling element to be connected with the power and torque transmission hose at additional places, and possibly also over its entire length. The pulling element can be a rope, such as a metallic rope, a wire, fiber or fiber bundle. The aforementioned list, however, is to be understood only as examples; it is also possible, of course, to use other pulling elements, with which the same purpose, a stiffening of the pulling strength of the power and torque transmission hose, can be achieved.

These and other advantages are presented by the following description with reference to the accompanying drawings. Reference to drawings in the description serves to reinforce the description and to facilitate comprehension of the object. Objects or parts of objects that are essentially identical or similar can be labeled with identical reference numbers. The drawings are merely schematic depictions of embodiments of the invention.

### BRIEF DESCRIPTION OF THE DRAWINGS

The drawings are as follows.

FIG. 1 is a perspective view of the microsurgical instrument.

FIG. 2 is a perspective detail view of the microsurgical instrument.

FIG. 3 is a longitudinal section of the distal end portion of the shaft.

FIG. 4 is another longitudinal section of the distal end portion of the shaft.

FIG. 5 is a cross-section of the shaft in the bendable shaft portion.

FIG. 6 is a detail view of the cross-section of the shaft in the bendable shaft portion.

### DETAILED DESCRIPTION

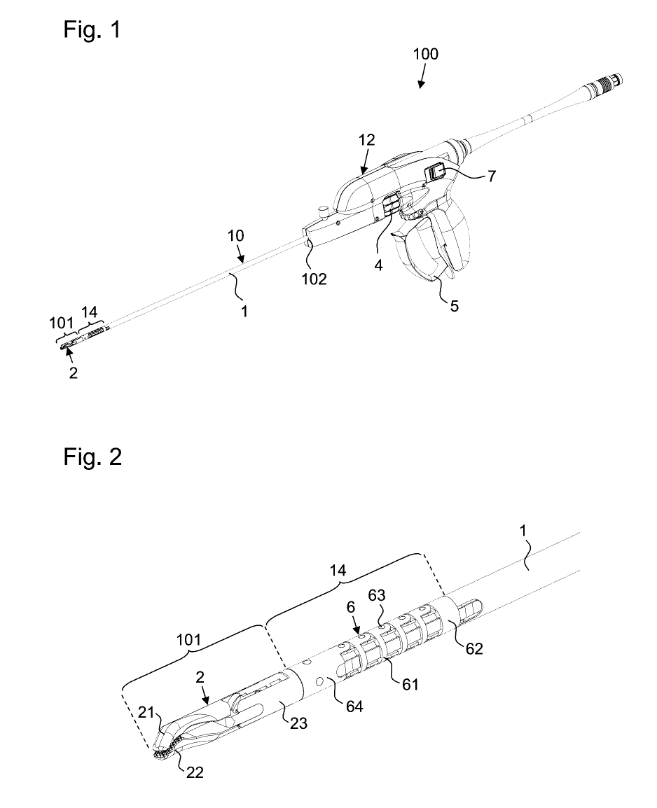

The invention relates to a work device 10 for a microsurgical instrument 100, as shown perspectivally in FIGS. 1 and 2. The work device 10 consists here of a work apparatus 2 and connecting shaft 1. The microsurgical instrument 100 consists essentially of the work apparatus 2 and connecting shaft 1. The microsurgical instrument 100 consists essentially of a handle 12 and the coupled work device 10.

The functions of the work device 10 can be controlled by means of the handle 12, or more precisely its actuation elements 4,5,7. In the present case the functions consist of: rotating the work apparatus 2 about the longitudinal axis of the shaft 1, opening/closing the arms 21,22 (see FIG. 2) of the work apparatus 2 and bending the bendable shaft portion 14. The handle 12 has three actuation elements 4,5,7 (FIG. 1), so that the work apparatus 2 can be rotated about the longitudinal axis by the first (manual) actuation element 4, the arms 21,22 can be opened and closed by means of the second (manual) actuation element 5, and the actuation element 7, which is an electrical actuation element 7, can activate a power drive, by which the shaft 1 can be bent in the bendable shaft segment 14.

The tool end 101 is at a distal end of the work apparatus 10, in which the work apparatus 2 is coupled with the shaft 1, which serves to provide mechanical connection with the handle 12. The shaft 1, in turn, is coupled at its proximal coupling portion 102 with the handle 12. The shaft 1 is hollow and, for example, configured as a tube of a metal or plastic. Then a power and torque transmission apparatus is attached, which serves to transmit power and torque from the handle 12 to the work apparatus 2. The power and torque transmission apparatus is, for example, a tube or a rod. In the bendable shaft portion 14, the power and torque transmission apparatus, however, is configured as a power and torque transmission tube 3 (see FIG. 4), which is pliable to enable it to follow the bend.

The bendable shaft portion 14 is depicted in detail in FIG. 2, The bend is achieved by several joints 6, which consist of links 61, 62, 64. The links 61, 62, 64 are connected to one another in pivotable manner by an axis 63, here a rivet shaft 63. A proximal connecting link 63 is positioned on the proximal end (to the right in the illustration) of the bendable shaft segment 14, and a distal connection link 64 is on the distal end of the bendable shaft segment 14, so that the connection links 62,64 in each case provide the junction to the shaft 1 or work apparatus 2. Between the proximal connection link 62 and the distal connection link 62 are the connection links 61, here four pieces. In an alternative embodiment, not shown, there can of course be more or fewer connection links 61, depending on the foreseeable articulation angle and radius.

The work apparatus 2 had two openable and closable arms 21,22 and is configured here as a grasping forceps, while in other embodiments it can be, for example, a needle holder, scissors or the like. The two arms 21, 22 are mounted in pivotable manner in a base 23 of the work apparatus 2. The base 23 of the work apparatus 2, in turn, is mounted so that it can rotate about the longitudinal axis of the shaft 1 with respect to the shaft 1 or to the distal connection link 64.

The illustrated work device 10 is an electro-surgical work device 10, which makes it possible to feed a flow of current between the two arms 21, 22.

For this purpose, running inside the power and torque transmission apparatus and the power and torque transmission hose 3 are two electrical connection lines 32, which are each conductively connected with one of the arms 21, 22; see FIG, 3. A (HF) current passes then between the arms 21, 22 through the tissue grasped by the arms 21,22. In an embodiment that is not shown in an illustration, the work device 10 can take the form of a work device 10 of a unipolar instrument; that is, only one cable runs through the power and torque transmission apparatus and the power and torque transmission hose 3, and the current flows in this case between the arms 21, 22 and a neutral electrode that is secured on the patient.

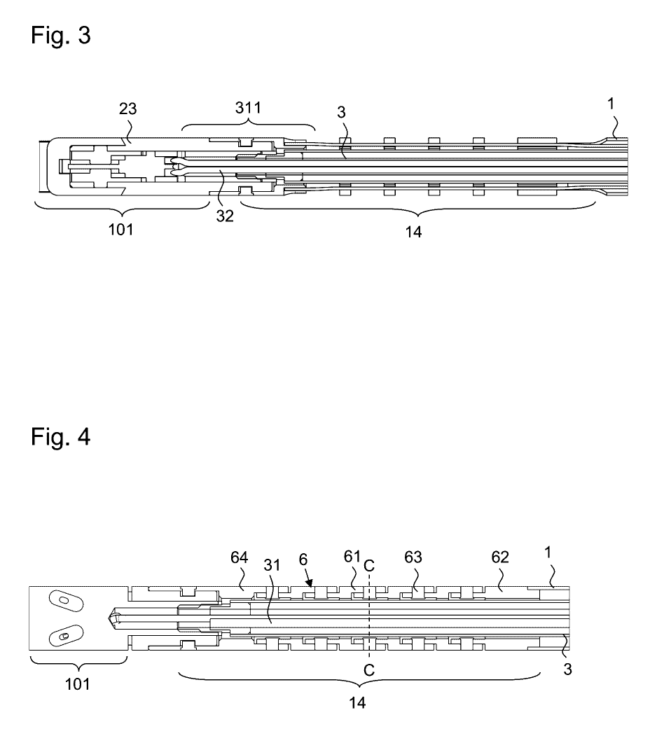

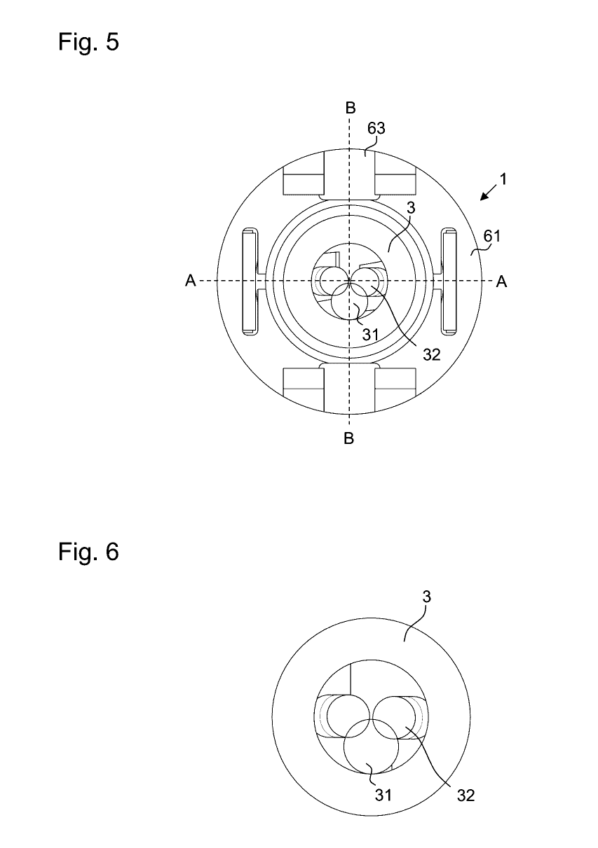

FIGS. 3 and 4 show two longitudinal sections of the distal end portion of the work device 10, while the sectional planes are indicated in FIG. 5. FIG. 3 depicts the section A-A, and FIG. 4 the section B-B.

Clearly indicated in FIG. 3, in particular, are the electrical connection lines 32 extending to the distal tool end 101. At their distal ends (to the left in the illustration) the electrical connection lines 32 are each connected conductively with one of the arms 21, 22 (see FIG. 2). Visible in the bendable shaft segment 14 is the power and torque transmission hose 3, in which the electrical connection lines 32 run. In order to feed the forces and moments, the power and torque transmission hose 3 at its distal end 311 is operatively coupled with the work apparatus 2.

The mechanical structure of the bendable shaft segment 14 can be clearly recognized in FIG, 4, whose sectional plane is turned 90° to FIG. 3. The linking elements 61, 62, 63, which make up the joints 6, are pivotably connected with one another by the axles 63 or rivet shafts 63. The power and torque transmission hose 3 runs inside the links 61, 62, 63.

The power and torque transmission hose 3 is made rigid to extend it in the pulling direction (longitudinal axis) by means of a pulling element 31, which in the distal end portion 311 (see FIG. 3) and the proximal end portion (not illustrated) is welded in each case with the power and torque transmission tube 3. The power and torque transmission tube 3 is thereby advantageously freed from pulling forces over a majority of its length, resulting in greater pulling resistance and thus providing the work device 10 greater attainable jaw strengths and an improved force feedback.

Finally, in FIGS. 5 and 6 a cross-section of the work device is shown, which was generated in relation to the sectional plane C-C (see FIG. 4). FIG. 6 presents a detail view that shows only the power and torque transmission hose 3. The sectional plane is accordingly in the bendable shaft portion 14, in which the power and torque transmission hose 3 is fed through the links 61. Consequently the inside of the power and torque transmission hose 3 is easily recognizable, in particular the pulling reinforcement 31 and the two electrical connection lines 32 fed into it.

The drawn overlapping of the power and torque transmission hose 3 and the electrical connection lines 32 are not significant and result merely from the drawing derivation. In the sectional plane C-C shown here, the electrical connection lines 32 and the power and torque transmission hose 3 do not overlap, but instead are each positioned on the inner cross-section of the power and torque transmission hose 3.

The power and torque transmission hose 3 can be constructed, for example, of spiral-shaped wound strips, which are connected on their respective longitudinal axes by a flange which forms protruding ribs (not shown in the illustration). Between the ribs formed by the flange there are indents, in each of which a surrounding wire coil can be installed to increase torsion resistance and to reduce the risk of unsteadiness. The power and torque transmission hose 3 can also be constructed on other basic principles, of course, for instance as a pliable axle or a shower hose, which is then stiffened with the pulling element 31.

1. A work device for a microsurgical instrument having:
a proximal coupling portion, which can be coupled with a handle,
whereby a hollow shaft extends from the coupling portion to a distal tool end on which is positioned a jaw-shaped work apparatus, said work apparatus having two arms which can pivot with respect to one another, and said work apparatus being mounted so that it can rotate about the longitudinal axis of the shaft,
and whereby a power and torque transmission apparatus, inside the shaft, extends from the coupling portion to the distal tool end, said apparatus being configured along one longitudinal portion as a power and torque transmission hose, so that the power and torque transmission apparatus is operatively connected with the work apparatuses to transmit a rotary motion and by opening and closing forces,
wherein the power and torque transmission hose is stiffened at least in one longitudinal portion with at least one pulling element running in the longitudinal direction.

2. The work device according to claim 1, wherein the pulling element
is positioned inside, preferably on the inner diameter, or outside, preferably on the outer diameter, of the power and torque transmission hose and/or
is connected, at least in one proximal and one distal end portion of the power and torque transmission hose, with the power and torque transmission hose, preferably by welding, and/or
is a rope, preferably a metallic rope, a wire, a fiber or fiber bundle.

3. The work device according to claim 1, wherein
the work apparatus is connected by a joint with the shaft, by which the work apparatus can turn about a rotary axis running perpendicular to the longitudinal axis of the shaft, and/or
the shaft comprises in a bendable shaft segment at least one joint, preferably two or more joints, by which it can turn about a rotary axis running perpendicular to the longitudinal axis of the shaft and/or in at least one longitudinal portion is pliable, so that the power and torque transmission apparatus, preferably in the bendable shaft segment, is configured as a power and torque transmission hose,
the shaft can be longitudinally adjusted, preferably telescoped.

4. The work device according to claim 1, wherein the work device is an electro-surgical work device, whereby at least one electrical connection line extends along the shaft from the coupling portion to the distal tool end and is electrically connected with at least one of the arms.

5. The work device according to claim 4, wherein the at least one electrical connection line runs in the interior of the power and torque transmission hose, preferably at a peripheral position opposite the pulling element or centered.

6. A microsurgical instrument, which comprises a proximal handle having at least one actuation element and a distal work device, which is operatively coupled with a corresponding coupling apparatus of the handle by its coupling portion, wherein the work device includes:
a proximal coupling portion, which can be coupled with a handle,
whereby a hollow shaft extends from the coupling portion to a distal tool end on which is positioned a jaw-shaped work apparatus, said work apparatus having two arms which can pivot with respect to one another, and said work apparatus being mounted so that it can rotate about the longitudinal axis of the shaft,
and whereby a power and torque transmission apparatus, inside the shaft, extends from the coupling portion to the distal tool end, said apparatus being configured along one longitudinal portion as a power and torque transmission hose, so that the power and torque transmission apparatus is operatively connected with the work apparatus to transmit a rotary motion and by opening and closing forces,
where the power and torque transmission hose is stiffened at least in one longitudinal portion with at least one pulling element running in the longitudinal direction.

7. The microsurgical instrument according to claim 6, wherein the handle:
is configured as a pistol grip or pincers grip and/or
comprises at least two mechanical actuation elements, a first actuation element to exert torque on the power and torque transmission apparatus of the work device and a second actuation element to exert a force in the longitudinal direction of the power and torque transmission apparatus of the work device.

8. The microsurgical instrument according to claim 7, wherein at least one of the mechanical actuation elements:
is a manual actuation element, preferably a rotary wheel, a lever, a pushbutton, and/or
is an actuation element by which a power drive apparatus can be actuated, which is configured to act on the power and torque transmission apparatus so that the power drive apparatus is preferably a rotary and/or linear power drive, especially preferably an electrical or pneumatic rotary and/or linear power drive.

9. A power and torque transmission hose for a work device having:
a proximal coupling portion, which can be coupled with a handle,
whereby a hollow shaft extends from the coupling portion to a distal tool end on which is positioned a jaw-shaped work apparatus, said work apparatus having two arms which can pivot with respect to one another, and said work apparatus being mounted so that it can rotate about the longitudinal axis of the shaft,
and whereby a power and torque transmission apparatus, inside the shaft, extends from the coupling portion to the distal tool end, said apparatus being configured along one longitudinal portion as a power and torque transmission hose, so that the power and torque transmission apparatus is operatively connected with the work apparatus to transmit a rotary motion and by opening and closing forces,
wherein the power and torque transmission hose is stiffened at least in one longitudinal portion with at least one pulling element running in the longitudinal direction; and
wherein the power and torque transmission hose, at least in a portion situated between its two ends, is stiffened by at last one pulling element running in the longitudinal direction of the power and torque transmission hose.

10. The power and torque transmission hose according to claim 9, wherein the pulling element
is positioned inside, preferably on the inner diameter, or outside, preferably on the outer diameter, of the power and torque transmission hose and/or
is connected, at least in a proximal and a distal end portion of the power and torque transmission hose with the power and torque transmission hose, preferably by welding, and/or
is a rope, preferably a metallic rope, a wire, a fiber or a fiber bundle.

11. The work device according to claim 2, wherein
the work apparatus is connected by a joint with the shaft, by which the work apparatus can turn about a rotary axis running perpendicular to the longitudinal axis of the shaft, and/or
the shaft comprises in a bendable shaft segment at least one joint, preferably two or more joints, by which it can turn about a rotary axis running perpendicular to the longitudinal axis of the shaft and/or in at least one longitudinal portion is pliable, so that the power and torque transmission apparatus, preferably in the bendable shaft segment, is configured as a power and torque transmission hose,
the shaft can be longitudinally adjusted, preferably telescoped.
